(19) United States
(12) Patent Application Publication
(10) Pub. No.: US 2007/0093505 A1
(43) Pub. Date: Apr. 26, 2007

(54) 2,3-Substituted 5,6-diaryl-pyrazine derivatives as cb1 modulators

(75) Inventors: Leifeng Cheng, Molndal (SE); Michael Wilstermann, Molndal (SE)

Correspondence Address: FISH & NEAVE IP GROUP;ROPES & GRAY LLP, ONE INTERNATIONAL PLACE, BOSTON, MA, 02110-2624, US

(73) Assignee: ASTRAZENECA AB, SODERTALJE (SE)

(21) Appl. No.: 10/561,033

(22) PCT Filed: Jun. 16, 2004
(86) PCT No.: PCT/SE04/00968

§ 371 Date: Dec. 16, 2005

(30) Foreign Application Priority Data
Jun. 19, 2003 (GB) 0314261.9

(51) Int. Cl. A61K 31/497; A61K 31/4965; C07D 413/02; C07D 403/02

(52) U.S. Cl. 514/255.05; 514/255.06; 544/405; 544/406; 544/230

(57) ABSTRACT

The present invention relates to 2,3-substituted-4,5-diarylpyrazine compounds and their compositions, processes for their preparation, and their use in the treatment of obesity and psychiatric and neurological disorders.

### FIELD OF INVENTION

The present invention relates to certain 4,5-diaryl-3-heterocyclylpyrazine-2-ester derivatives of formula I, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB₁ modulators (known as antagonists or inverse agonists) are useful in the treatment of obesity, psychiatric and neurological disorders (WO01/70700 and EP 656354). However, there is a need for CB₁ modulators with improved physicochemical properties and/or DMPK properties and/or pharmacodynamic properties.

Pyrazinecarboxamides are reported to possess antithrombotic properties (WO 92/02513). The compounds disclosed in this document are disclaimed from the compound claims of the present invention. 5,6-Diphenyl-2-pyrazinecarboxylic acid is disclosed in CH 458 361.

Co-pending application PCT/GB02/05742 discloses compounds of the general formula (A) 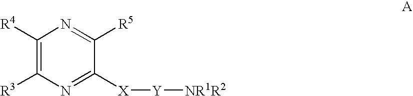
and pharmaceutically acceptable salts, prodrugs, solvates and crystalline forms thereof, in which
R¹ and R² independently represent:
a C₁₋₆alkyl group;
an (amino)C₁₋₄alkyl- group in which the amino is optionally substituted by one or more C₁₋₃alkyl groups;
an optionally substituted non-aromatic C₃₋₁₅carbocyclic group;
a (C₃₋₁₂cycloalkyl)C₁₋₃alkyl- group;
a group —(CH₂)ᵣ(phenyl)ₛ in which r is 0, 1, 2, 3 or 4, s is 1 when r is 0 otherwise s is 1 or 2 and the phenyl groups are optionally independently substituted by one, two or three groups represented by Z;
naphthyl;
anthracenyl;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
1-adamantylmethyl;
a group—(CH₂)ₜ Het in which t is 0, 1, 2, 3 or 4, and the alkylene chain is optionally substituted by one or more C₁₋₃alkyl groups and Het represents an aromatic heterocycle optionally substituted by one, two or three groups selected from a C₁₋₅alkyl group, a C₁₋₅alkoxy group or halo;
or R¹ represents H and R² is as defined above;
or R¹ and R² together with the nitrogen atom to which they are attached represent a saturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy or benzyl;
X is CO or SO₂;
Y is absent or represents NH optionally substituted by a C₁₋₃alkyl group;
R³ and R⁴ independently represent phenyl, thienyl or pyridyl each of which is optionally substituted by one, two or three groups represented by Z;
   Z represents a C₁₋₃alkyl group, a C₁₋₃alkoxy group, hydroxy, halo, trifluoromethyl, trifluoromethylthio, trifluoromethoxy, trifluoromethylsulphonyl, nitro, amino, mono or di C₁₋₃alkylamino, mono or di C₁₋₃alkylamido, C₁₋₃alkylsulphonyl, C₁₋₃alkoxycarbonyl, carboxy, cyano, carbamoyl, mono or di C₁₋₃alkyl carbamoyl, sulphamoyl and acetyl; and
   R⁵ is H, a C₁₋₃alkyl group, a C₁₋₃alkoxymethyl group, trifluoromethyl, a hydroxyC₁₋₃alkyl group, C₁₋₃alkoxycarbonyl, carboxy, cyano, carbamoyl, mono or di C₁₋₃alkylcarbamoyl, acetyl, or hydrazinocarbonyl of formula —CONHNR^{a}R^{b} wherein R^{a} and R^{b} are as previously defined for R¹ and R² respectively;
   with the proviso that when R¹ and R² together with the nitrogen atom to which they are attached represent 4methylpiperazin-1-yl or R¹ represents H and R² represents methyl or 1-benzylpiperidin-4-yl; X is CO; Y is absent and R⁵ is H; then R³ and R⁴ do not both represent 4-methoxyphenyl; and their use in the treatment of obesity, psychiatric and neurological disorders.

### DESCRIPTION OF THE INVENTION

The invention relates to a compound of formula (I) 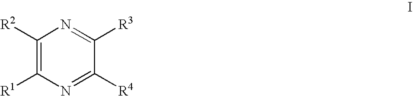
and pharmaceutically acceptable salts thereof, in which
R¹ and R² independently represent phenyl, thienyl or pyridyl each of which is independently optionally substituted by one or more groups represented by Z;
Z represents a C₁₋₈alkyl group, a C₁₋₆alkoxy group, hydroxy, halo, trifluoromethyl, trifluoromethylthio, trifluoromethoxy, trifluoromethylsulphonyl, nitro, mono or di C₁₋₃alkylamido, C₁₋₃alkylthio, C₁₋₃alkylsulphonyl, C₁₋₃alkylsulphonyloxy, C₁₋₃alkoxycarbonyl, carboxy, cyano, carbamoyl, mono or di C₁₋₃alkyl carbamoyl, sulphamoyl, acetyl, an aromatic heterocyclic group which is optionally substituted by one or more halo, C₁₋₄alkyl, trifluoromethyl or trifluoromethoxy and a saturated or partially unsaturated 5 to 8 membered heterocyclic group containing one or more heteroatoms selected from nitrogen, oxygen or sulphur wherein the heterocyclic group is optionally substituted by one or more C₁₋₃alkyl groups, hydroxy, fluoro, benzyl or an amino group —NR^{x}R^{y} in which R^{x} and R^{y} independently represent H or C₁₋₄alkyl;
R³ and R⁴ independently represent a group of formula (CH₂)ₙCOOR⁷
in which n is 0, 1, 2, 3 or 4; and R⁷ represents a C₄₋₁₂alkyl group, a C₃₋₁₂cycloalkyl group or a (C₃₋₁₂cycloalkyl)C₁₋₃alkyl- group each of which is optionally substituted by one or more of the following: a C₁₋₆-alkyl group; fluoro, amino or hydroxy, or
R⁷ represents a group —(CH₂)ₐphenyl in which a is 0, 1, 2, 3 or 4 and the phenyl group is optionally substituted by one or more groups represented by Z which may be the same or different or
R⁷ represents a saturated or partially unsaturated 5 to 8 membered heterocyclic group containing one or more of the of the following: oxygen, sulphur or nitrogen; wherein the heterocyclic group is optionally substituted by one or more C₁₋₃alkyl groups, C₁₋₃acyl groups, hydroxy, amino or benzyl; or
R³ and R⁴ independently represent a group of formula —(CH₂)ₒ—O—(CH₂)ₚ—R⁸ in which o and p independently represent an integer 0, 1, 2, 3 or 4 with the proviso that neither R³ or R⁴ is methoxy, and R⁸ represents a C₁₋₁₂alkyl group or R⁸ represents phenyl optionally independently substituted by one or more Z groups or R⁸ represents an aromatic heterocyclic group or a saturated or partially unsaturated 5 to 8 membered heterocyclic group containing one or more of one following: oxygen, sulphur or nitrogen wherein each of these rings is optionally substituted by one or more groups represented by Z which may be the same or different;
R³ and R⁴ independently represent a C₁₋₁₂alkyl group optionally substituted by one or more fluoro, hydroxy, or amino, provided that if R³ is C₁₋₄alkyl then R⁴ cannot be C₁₋₄alkyl or q cannot be 0 in R⁴, or
R³ and R⁴ independently represent a group of formula —(CH₂)_{q}R⁹ in which q is 0, 1, 2, 3 or 4, provided that if q is 0 in R³ then q cannot be 0 R⁴, and vice versa, R⁹ represents a C₃₋₁₂cycloalkyl group, phenyl, an aromatic heterocyclic group or a saturated or partially unsaturated 5 to 12 membered heterocyclic group containing one or more of one following: oxygen, sulphur or nitrogen, wherein each of these rings is optionally substituted by one or more groups represented by Z which may be the same or different or each of these rings is substituted by phenyl which optionally substituted by more C₁₋₄alkyl, a C₁₋₄alkoxy, hydroxy, halo or trifluoromethyl.

R³ and R⁴ independently represent a group of formula —(CH₂)ₘ—O—(CO)—R¹⁰ in which m represents an integer 0, 1, 2, 3 or 4, in which R¹⁰ represents a C₁₋₁₂alkyl group optionally substituted by one or more fluoro, hydroxy, or amino or R¹⁰ represents a group of formula —(CH₂)_{q}R⁹ in which

q and R⁹ is as previously described;

or

R³ and R⁴ are identical and represent a group of formula CONR¹¹R¹²

in which

R¹¹ and R¹² independently represent a C₁₋₆alkyl group;

an (amino)C₁₋₄alkyl- group in which the amino is optionally substituted by one or more C₁₋₃alkyl groups;

a (C₃₋₁₂cycloalkyl)(CH₂)_{g}— group wherein g is 0, 1, 2 or 3 wherein the cycloalkyl is optionally substituted by one or more fluoro, hydroxy, C₁₋₃alkyl, C₁₋₃alkoxy, C₁₋₃alkoxycarbonyl, trifluoromethyl, amino or trifluoromethoxy;

a group —(CH₂)ᵣ(phenyl)ₛ in which r is 0, 1, 2, 3 or 4, s is 1 when r is 0 otherwise s is 1 or 2 and the phenyl groups are optionally independently substituted one or more groups represented by Z;

naphthyl;

anthracenyl;

a saturated or partially unsaturated 5 to 8 membered heterocyclic group containing one or more heteroatoms selected from nitrogen, oxygen or sulphur wherein the heterocyclic group is optionally substituted by one or more C₁₋₃alkyl groups, hydroxy, fluoro, trifluoromethyl, benzyl or an amino group —NR^{x}R^{y} in which R^{x} and R^{y} independently represent H or C₁₋₄alkyl; 1-adamantylmethyl;

a group—(CH₂)ₜ Het in which t is 0, 1, 2, 3 or 4, and the alkylene chain is optionally substituted by one or more C₁₋₃alkyl groups and Het represents an aromatic heterocyclic group optionally substituted by one, two or three groups selected from a C₁₋₅alkyl group, a C₁₋₅alkoxy group or halo;

or R¹¹ represents H and R¹² is as defined above;

or R¹¹ and R¹² together with the nitrogen atom to which they are attached represent a saturated or partially unsaturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy, fluoro, trifluoromethyl, trifluoromethoxy, benzyl, C₁₋₆alkanoyl or an amino group —NR^{x}R^{y} in which R^{x} and R^{y} independently represent H or C₁₋₄alkyl;

with the provisos that

1) when R³ and R⁴ are both a group of formula CONR¹¹R¹² then they do not represent carbamoyl, or mono or di C₁₋₃alkylcarbamoyl and

2) when R¹, R² and R³ each represent phenyl then R⁴ is not benzyl.

3) when one of R³ or R⁴ is C₁₋₄alkyl then the other is not a group —(CH₂)_{q}R⁹ in which q is 0.

It will be understood that where a substituent Z is present in more than one group that these substituents are independently selected and may be the same or different.

The term aromatic heterocyclic group means an aromatic 5-, 6-, or 7-membered monocyclic ring or a 9- or 10-membered bicyclic ring, with up to five ring heteroatoms selected from oxygen, nitrogen and sulfur. Suitable aromatic heterocyclic groups include, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridonyl, pyridazinyl, pyridazonoyl pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl, preferably furyl, pyrrolyl, thienyl, oxazolyl, isoxazolyl, imidazolyl, pyrazolyl, oxazolyl thiazolyl, isothiazolyl, oxadiazolyl, thiadiazolyl, triazolyl, tetrazolyl, pyridyl, pyridazinyl, pyrimidinyl, pyrazinyl or 1,3,5-triazenyl and more preferably pyrrolyl, thienyl, imidazolyl, oxazolyl or pyridyl.

Suitable saturated or partially unsaturated 5 to 12 membered heterocyclic group containing one or more heteroatoms selected from nitrogen, oxygen or sulphur may be monocyclic or bicyclic and includes spiro bicyclic groups for example oxiranyl, oxetanyl, tetrahydrofuranyl, tetrahydropyranyl, 2,3-dihydro-1,3-thiazolyl, 1,3-thiazolidinyl, 4,5-dihydrooxazol-2-yl, (3-oxa-1-azaspiro[4.4]non-1-en-2-yl), pyrrolinyl, pyrrolidinyl, morpholinyl, tetrahydro-1,4-thiazinyl, 1-oxotetrahydrothienyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl or tetrahydropyrimidinyl, preferably tetrahydrofuranyl, tetrahydropyranyl, pyrrolidinyl, morpholinyl, piperidinyl or piperazinyl, more preferably tetrahydrofuran-3-yl, tetrahydropyran-4-yl, pyrrolidin-3-yl, morpholino, piperidino, piperidin-4-yl or piperazin-1-yl.

Further values of R¹, R², R³ and R⁴ in compounds of formula I now follow. It will be understood that such values may be used where appropriate with any of the definitions, claims or embodiments defined hereinbefore or hereinafter.

In a first group of compounds of formula, R¹ and R² are phenyl optionally substituted by one or more groups Z.

In a second group of compounds of formula I, R¹ and R² are both 4-chlorophenyl.

In a third group of compounds of formula I, R³ and R⁴ independently represent a group of formula COOR⁷ in which R⁷ is a C₄₋₈alkyl group.

In a fourth group of compounds of formula I, R³ represents a group of formula COOR⁷ in which R⁷ is a C₄₋₈alkyl group and R⁴ represents a group of formula —(CH₂)ₒ—O—(CH₂)ₚ—R⁸ in which o and p independently represent an integer 0, 1, 2, 3 or 4 R⁸ represents phenyl optionally independently substituted by one or more Z groups.

In a fifth group of compounds of formula I, R³ and R⁴ are identical and each represent a group of formula CONR¹¹R¹² in which R¹¹ and R¹² are as previously defined with provisos.

In a sixth group of compounds of formula I, R³ and R⁴ each represent a group of formula CONR¹¹R¹² in which R¹¹ and R¹² together with the nitrogen atom to which they are attached represent piperidino.

In a seventh group of compounds of formula I, R³ represents a group of formula COOR⁷ in which R⁷ is a C₄₋₈alkyl group and R⁴ represents a group of formula R³ and R⁴ independently represent a group of formula —(CH₂)ₘ—O—(CO)—R¹⁰ in which m represents an integer 0, 1, 2, 3 or 4, in which R¹⁰ represents a C₁₋₁₂alkyl group optionally substituted by one or more fluoro, hydroxy, or amino or R¹⁰ represents phenyl optionally substituted by one or more groups represented by Z which may be the same or different.

In an eighth group of compounds, which is a sub group of the each of the first, second and third groups R³ and R⁴ are identical.

A particular group of compounds of formula I is represented by formula II 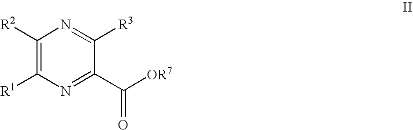
in which R¹ and R² are both 4-chlorophenyl;
R³ represents dihydrooxazolyl, (3-oxa-1-azaspiro[4.4]nonenyl), oxazolyl or tetrazol-2-ylmethyl optionally substituted by phenyl or a C₁₋₄alkyl group; and
R⁷ represents a C₄₋₁₂alkyl group, a C₃₋₁₂cycloalkyl group or a (C₃₋₁₂cycloalkyl)C₁₋₃alkyl group each of which is optionally substituted by one or more of the following: a C₁₋₆alkyl group; fluoro, amino or hydroxy.

Particularly R⁷ is tert-butyl.

Particularly R³ represents (4,4-dimethyl-4,5-dihydrooxazol-2-yl), (3-oxa-1-azaspiro[4.4]non-1-en-2-yl), (4-methyl-4,5-dihydrooxazol-2-yl), (4-methyloxazol-2-yl), (4-phenyl-4,5-dihydrooxazol-2-yl), (4-phenyloxazol-2-yl), (5-phenyl-4,5-dihydrooxazol-2-yl) or 3-(2H-tetrazol-2-ylmethyl).

Another aspect of the invention relates to the use a compound of formula (Ia) and pharmaceutically acceptable salts thereof, in the preparation of a medicament for the treatment or prophylaxis of obesity, psychiatric disorders such as psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, and neurological disorders such as dementia, neurological disorders, Parkinson's Disease, Huntington's Chorea and Alzheimer's Disease, immune, cardiovascular, reproductive and endocrine disorders, septic shock, diseases related to the respiratory and gastrointestinal systems, and extended abuse, addiction and/or relapse indications.

Formula Ia has the following general formula: 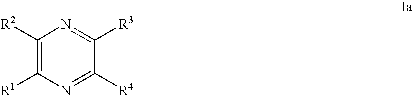
in which R¹ and R² independently represent phenyl, thienyl or pyridyl each of which is independently optionally substituted by one or more groups represented by Z;

Z represents a C₁₋₈alkyl group, a C₁₋₆alkoxy group, hydroxy, halo, trifluoromethyl, trifluoromethylthio, trifluoromethoxy, trifluoromethylsulphonyl, nitro, mono or di C₁₋₃alkylamido, C₁₋₃alkylsulphonyl, C₁₋₃alkylsulphonyloxy, C₁₋₃alkoxycarbonyl, carboxy, cyano, carbamoyl, mono or di C₁₋₃alkyl carbamoyl, sulphamoyl, acetyl, an aromatic heterocyclic group which is optionally substituted by one or more halo, C₁₋₄alkyl, trifluoromethyl or trifluoromethoxy and a saturated or partially unsaturated 5 to 8 membered heterocyclic group containing one or more heteroatoms selected from nitrogen, oxygen or sulphur wherein the heterocyclic group is optionally substituted by one or more C₁₋₃alkyl groups, hydroxy, fluoro, benzyl or an amino group —NR^{x}R^{y} in which R^{x} and R^{y} independently represent H or C₁₋₄alkyl;

R³ and R⁴ independently represent a group of formula (CH₂)ₙCOOR⁷

in which n is 0, 1, 2, 3 or 4; and R⁷ represents a C₁₋₁₂alkyl group, a C₃₋₁₂cycloalkyl group or a (C₃₋₁₂cycloalkyl)C₁₋₃alkyl- group each of which is optionally substituted by one or more of the following: a C₁₋₆alkyl group; fluoro, amino or hydroxy, or

R⁷ represents a group —(CH₂)ₐphenyl in which a is 0, 1, 2, 3 or 4 and the phenyl group is optionally substituted by one or more groups represented by Z which may be the same or different or

R⁷ represents a saturated or partially unsaturated 5 to 8 membered heterocyclic group containing one or more of the of the following: oxygen, sulphur or nitrogen; wherein the heterocyclic group is optionally substituted by one or more C₁₋₃alkyl groups, C₁₋₃acyl groups, hydroxy, amino or benzyl; or

R³ and R⁴ independently represent a group of formula —(CH₂).—O—(CH₂)ₚ—R⁸ in which o and p independently represent an integer 0, 1, 2, 3 or 4 and R⁸ represents a C₁₋₁₂alkyl group or R⁸ represents phenyl optionally independently substituted by one or more Z groups or R⁸ represents an aromatic heterocyclic group or a saturated or partially unsaturated 5 to 8 membered heterocyclic group containing one or more of one following: oxygen, sulphur or nitrogen wherein each of these rings is optionally substituted by one or more groups represented by Z which may be the same or different;

R³ and R⁴ independently represent a C₁₋₁₂alkyl group optionally substituted by one or more fluoro, hydroxy, or amino; or

R³ and R⁴ independently represent a group of formula —(CH₂)_{q}R⁹ in which q is 0, 1, 2, 3 or 4 and R⁹ represents a C₃₋₁₂cycloalkyl group, phenyl, an aromatic heterocyclic group or a saturated or partially unsaturated 5 to 8 membered heterocyclic group containing one or more of one following: oxygen, sulphur or nitrogen wherein each of these rings is optionally substituted by one or more groups represented by Z which may be the same or different; or

R³ and R¹ independently represent a group of formula —(CH₂)ₘ—O—(CO)—R¹⁰ in which m represents an integer 0, 1, 2, 3 or 4, in which R¹⁰ represents a C₁₋₁₂alkyl group optionally substituted by one or more fluoro, hydroxy, or amino or R¹⁰ represents a group of formula —(CH₂)_{q}R⁹ in which

q and R⁹ is as previously described;

or

R³ and R⁴ independently represent a group of formula CONR¹¹R¹²

in which

R¹¹ and R¹² independently represent a C₁.alkyl group;

an (amino)C₁₋₄alkyl- group in which the amino is optionally substituted by one or more C₁₋₃alkyl groups;

a (C₃₋₁₂cycloalkyl)(CH₂)_{g}— group wherein g is 0,1, 2 or 3 wherein the cycloalkyl is optionally substituted by one or more fluoro, hydroxy, C₁₋₃alkyl, C₁₋₃alkoxy, C₁₋₃alkoxycarbonyl, trifluoromethyl, amino or trifluoromethoxy;

a group —(CH₂)ᵣ(phenyl)_{g} in which r is 0, 1, 2, 3 or 4, s is 1 when r is 0 otherwise s is 1 or 2 and the phenyl groups are optionally independently substituted one or more groups represented by Z;

naphthyl;

anthracenyl;

a saturated or partially unsaturated 5 to 8 membered heterocyclic group containing one or is more heteroatoms selected from nitrogen, oxygen or sulphur wherein the heterocyclic group is optionally substituted by one or more C₁₋₃alkyl groups, hydroxy, fluoro, trifluoromethyl, benzyl or an amino group —NR^{x}R^{y} in which R^{x} and R^{y} independently represent H or C₁₋₄alkyl; 1-adamantylmethyl;

a group—(CH₂)ₜ Het in which t is 0, 1, 2, 3 or 4, and the alkylene chain is optionally substituted by one or more C₁₋₃alkyl groups and Het represents an aromatic heterocyclic group optionally substituted by one, two or three groups selected from a C₁₋₅-alkyl group, a C₁₋₅alkoxy group or halo;

or R¹¹ represents H and R¹² is as defined above;

or R¹¹ and R¹² together with the nitrogen atom to which they are attached represent a saturated or partially unsaturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy, fluoro, trifluoromethyl, trifluoromethoxy, benzyl, C₁₋₆alkanoyl or an amino group —NR^{x}R^{y} in which R^{x} and R^{y} independently represent H or C₁₋₄alkyl;

with the proviso that when one of R³ and R⁴ is a C₁₋₃alkyl group, a C₁₋₃alkoxymethyl group, trifluoromethyl, a hydroxyC₁₋₃alkyl group, C₁₋₃alkoxycarbonyl, carboxy, carbamoyl, or mono or di C₁₋₃alkylcarbamoyl then the other does not represent a group of formula CONR¹¹R¹².

In compounds of formula Ia the following two paragraphs apply.

The term aromatic heterocyclic group means an aromatic 5-, 6-, or 7-membered monocyclic ring or a 9- or 10-membered bicyclic ring, with up to five ring heteroatoms selected from oxygen, nitrogen and sulfur. Suitable aromatic heterocyclic groups include, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl. Preferably furyl, pyrrolyl, thienyl, oxazolyl, isoxazolyl, imidazolyl, pyrazolyl, oxazolyl thiazolyl, isothiazolyl, oxadiazolyl, thiadiazolyl, triazolyl, tetrazolyl, pyridyl, pyridazinyl, pyrimidinyl, pyrazinyl or 1,3,5-triazenyl and more preferably pyrrolyl, thienyl, imidazolyl, oxazolyl or pyridyl.

Suitable saturated or partially unsaturated 5 to 8 membered heterocyclic group containing one or more heteroatoms selected from nitrogen, oxygen or sulphur include, for example oxiranyl, oxetanyl, tetrahydrofuranyl, tetrahydropyranyl, 2,3-dihydro-1,3-thiazolyl, 1,3-thiazolidinyl, pyrrolinyl, pyrrolidinyl, morpholinyl, tetrahydro-1,4-thiazinyl, 1-oxotetrahydrothienyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl or tetrahydropyrimidinyl, preferably tetrahydrofuranyl, tetrahydropyranyl, pyrrolidinyl, morpholinyl, piperidinyl or piperazinyl, more preferably tetrahydrofuran-3-yl, tetrahydropyran-4-yl, pyrrolidin-3-yl, morpholino, piperidino, piperidin-4-yl or piperazin-1-yl.

It will be understood that where a substituent Z is present in more than one group that these substituents are independently selected and may be the same or different.

“Pharmaceutically acceptable salt”, where such salts are possible, includes both pharmaceutically acceptable acid and base addition salts.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention. All tautomers, where possible, are included within the scope of the invention.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl and t-butyl. Preferred alkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halogen” shall mean fluorine, chlorine, bromine or iodine.

Specific compounds of the invention are one or more of the following:
2,3-bis(4-chlorophenyl)-5,6-bis(piperidin-1-ylcarbonyl)pyrazine,
bis-2,3-(tert-butoxy)-5,6-bis(4-chlorophenyl)pyrazine-2,3-dicarboxylate,
5,6-bis(4-chlorophenyl)-3-(4,4-dimethyl-4,5-dihydrooxazol-2-yl)-pyrazine-2-carboxylic acid tert-butylester,
5,6-bis(4-chlorophenyl)-3-(3-oxa-1-azaspiro[4.4]non-1-en-2-yl)-pyrazine-2-carboxylic acid tert-butylester,
5,6-bis(4-chlorophenyl)-3-(4-methyl-4,5-dihydrooxazol-2-yl)-pyrazine-2-carboxylic acid tert-butylester,
5,6-bis(4-chlorophenyl)-3-(4-methyloxazol-2-yl)-pyrazine-2-carboxylic acid tert-butylester,
5,6-bis(4-chlorophenyl)-3-(4-phenyl-4,5-dihydrooxazol-2-yl)-pyrazine-2-carboxylic acid tert-butylester)
5,6-bis(4-chlorophenyl)-3-(4-phenyloxazol-2-yl)-pyrazine-2-carboxylic acid tert-butylester,
5,6-bis(4-chlorophenyl)-3-(5-phenyl-4,5-dihydrooxazol-2-yl)-pyrazine-2-carboxylic acid tert-butylester,
tert-butyl 5,6-bis(4-chlorophenyl)-3-(2H-tetrazol-2-ylmethyl)pyrazine-2-carboxylate and pharmaceutically acceptable salts thereof.
   Methods of Preparation

The compounds of the invention may be prepared as outlined below according to any of the following methods. However, the invention is not limited to these methods, the compounds may also be prepared as described for structurally related compounds in the prior art.

Compounds of formula I in which R¹ and R² are as previously defined and R⁴ is a group COOR⁴ and R³ is CONR¹¹R¹² may be prepared by reacting a compound of formula III 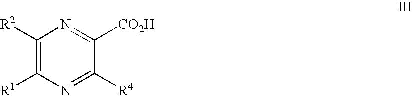
in which R¹, R² and R⁴ are as defined immediately previously with an amine of formula IV
R¹¹R¹²NH₂ IV
in which R¹¹ and R¹² are as previously defined in an inert solvent, for example dichloromethane, in the presence of a coupling agent, for example a carbodiimide, e.g., 1-(3-dimethylaminopropyl)-3-ethylcarbodiimide, and optionally in the presence of a catalyst, for example a basic catalyst, e.g., 4-dimethylaminopyridine, at a temperature in the range of −25° C. to 150° C.

Compounds of formula III may be prepared by reacting a compound of formula V 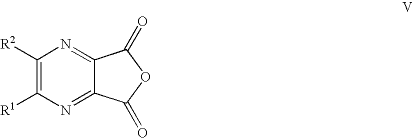
in which R¹ and R² are as previously defined with a compound of formula VI
R⁷OH VI
in which R⁷ is as previously defined in an inert solvent, for example acetonitrile, and optionally in the presence of a catalyst, for example a basic catalyst, e.g., 4-dimethylaminopyridine, at a temperature in the range of −25° C. to 150° C.

Compounds of formula I may also be prepared by reacting a compound of formula V with a compound of formula VI and then reacting the product directly with a compound of formula IV.

Compounds of formulae III, V and VII are commercially available or may be prepared by methods known to those skilled in the art. Certain compounds of formulae II, m, IV and V are novel and are claimed as a further aspect of the present invention as useful intermediates.

Compounds of formula V may be prepared by reacting a compound of formula VIII 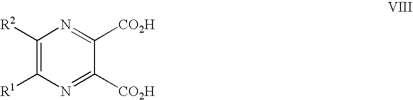
in which R¹ and R² are as previously defined with a dehydrating agent for example acetyl chloride at a temperature in the range of 0° C. to 150° C.

Other compounds of formula I may be prepared by analogous methods or by methods known to those skilled in the art.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

The expression “inert solvent” refers to a solvent which does not react with the starting materials, reagents, intermediates or products in a manner which adversely affects the yield of the desired product.

Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

Pharmacological Properties

The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders (e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight, which normally accompanies the cessation of smoking.

In another aspect the present invention provides a compound of formula I as previously defined for use as a medicament.

In a further aspect the present invention provides the use of a compound of formula I in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc.) withdrawal symptoms comprising administering a pharmacologically effective amount of a compound of formula I to a patient in need thereof.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc.) withdrawal symptoms comprising administering a pharmacologically effective amount of a compound of formula Ia to a patient in need thereof. Formula Ia is as defined above.

The compounds of the present invention are particularly suitable for the treatment of obesity, e.g., by reduction of appetite and body weight, maintenance of weight reduction and prevention of rebound.

Combination Therapy

The compounds of the invention may be combined with another therapeutic agent that is useful in the treatment of disorders associated with the development and progress of obesity such as hypertension, hyperlipidaemias, dyslipidaemias, diabetes and atherosclerosis. For example, a compound of the present invention may be used in combination with a compound that affects thermogenesis, lipolysis, fat absorption, satiety, or gut motility. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;

a cholesterol absorption antagonist;

a MTP (microsomal transfer protein) inhibitor;

a nicotinic acid derivative, including slow release and combination products;

a phytosterol compound;

probucol;

an anti-coagulant;

an omega-3 fatty acid;

another anti-obesity compound;

an antihypertensive compound for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, an AT-1 blocker, a saluretic, a diuretic or a vasodilator;

a Melanin concentrating hormone (MCH) antagonist;

a PDK inhibitor; or

modulators of nuclear receptors for example LXR, FXR, RXR, and RORalpha;

an SSRI;

a serotonin antagonist;

or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt thereof, in a first unit dosage form;

b) a compound from one of the other classes of compounds described in this combination section or a pharmaceutically acceptable salt, solvate, solvate of such a salt or a prodrug thereof; in a second unit dosage form; and

c) container means for containing said first and second dosage forms.

According to a further aspect of the present invention there is provided a kit comprising:

a) a compound of formula I, or a pharmaceutically acceptable salt thereof, together with a pharmaceutically acceptable diluent or carrier, in a first unit dosage form;

b) a compound from one of the other classes of compounds described in this combination section or a pharmaceutically acceptable salt, solvate, solvate of such a salt or a prodrug thereof, in a second unit dosage form; and

c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatorheic hepatitis, osteoarthritis and some cancers) and psychiatric and neurological conditions.

### EXAMPLES

Abbreviations

DCM—dichloromethane

DMF—dimethylformamide

DMAP—4-dimethylaminopyridine

EDC—1-(3-dimethylaminopropyl)-3-ethylcarbodiimide

TEA—triethylamine

TFA—trifluoroacetic acid

DMSO—dimethyl sulfoxide

DEA—diethylamine

PCC—pyridinium chlorochromate

DCM—dichloromethane

PyBOP—benzotriazol-1-yl-oxytri-pyrrolidinophosphonium hexafluorophosphate

HATU—O-(7-Azabenzotriazol-1-yl)-N,N′N′,N′-tetramethyluronium hexafluorophosphate

DAST—(diethyl amino)sulphur trifluoride

DIEA—N,N-diisopropylethylamine

DDQ—2,3-dichloro-5,6-dicyano-1,4-benzoquinone

HRMS—high resolution mass spectrometer

t triplet

s singlet

d doublet

q quartet

qvint quintet

m multiplet

br broad

bs broad singlet

dm doublet of multiplet

bt broad triplet

dd doublet of doublet

General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at ¹H frequencies of 300 and 500 MHz respectively. Chemical shifts are given in ppm with CDCl₃ as internal standard. CDCl₃ is used as the solvent for NMR unless otherwise stated. Purification was performed on a semipreparative HPLC with a mass triggered fraction collector, Shimadzu QP 8000 single quadrupole mass spectrometer equipped with 19×100 mm C8 column. The mobile phase used was, if nothing else is stated, acetonitrile and buffer (0.1 M N₄Ac:acetonitrile 95:5).

For isolation of isomers, a Kromasil CN E9344 (250×20 mm i.d.) column was used. Heptane:ethyl acetate:DEA 95:5:0.1 was used as mobile phase (1 ml/min). Fraction collection was guided using a UV-detector (330 nm).

### EXAMPLES OF THE INVENTION

### Example 1

### 2,3-bis(4-chlorophenyl)-5,6-bis(piperidin-1-ylcarbonyl)pyrazine

Oxalyl chloride (1.3 ml, 15 mmol) was added to a suspension of 5,6-bis(4-chlorophenyl)pyrazine-2,3-dicarboxylic acid, (589 mg, 1.51 mmol) in DCM (10 ml) and DMF (0.2 ml). After 10 minutes the solvent was removed in vacuo. The residue was retaken in dry toluene, filtrated through celite, and evaporated twice in order to completely remove excess oxalyl chloride. The residue was dissolved in DCM (20 ml) and a solution of piperidine (773 mg, 9.08 mmol) in DCM was added. After 1 h the reaction mixture was washed with hydrochloric acid (2 M), water and dried (magnesium sulfate). Evaporation of the solvent gave the target compound (43 mg, 54%).

¹H NMR (400 MHz) δ 7.40 (d, 4H), 7.30 (d, 4H), 3.74-3.69 (m, 4H), 3.49-3.43 (m, 4H), 1.72-1.64 (m, 12H).

MS m/z calcd for [C₂₈H₂₈Cl₂N₄O₂]H⁺523.1668, found 523.1655 (M+H)⁺.

### Example 2

### Bis-2,3-(tert-butoxy)-5,6-bis(4-chlorophenyl)pyrazine-2,3-dicarboxylate

Oxalyl chloride (1 ml, 11 mmol) was added to a suspension of 5,6-bis(4-chlorophenyl)pyrazine-2,3-dicarboxylic acid (210 mg, 0.54 mmol) in methylene chloride (5 ml) and then DMF (20 microliters) was added. After 1 hr a slightly turbid solution had formed which was filtered through celite and the solvent was removed in vacuo. Addition of toluene and evaporation of the solvent and mixing of the residue with t-butyl alcohol (1.05 g, 14 mmol) dissolved in pyridine (1 ml) and acetonitrile (5 ml). After 5 minutes the solvent was removed in vacuo and the residue was partioned between methylene chloride and 0.3 M KHSO4. Washing once more with KHSO4 and bicarbonate solution, drying (magnesium sulfate) and evaporation of the solvent gave a residue which was purified by preparative HPLC. The yield was 60 mg (22%)

¹H NMR (400 MHz, CDCl₃) δ7.46 (d, 4H), 7.31 (d, 4H), 1.64 (s, 18H).

MS m/z calcd for [C26H26N2O4C12]H⁺501.1348, found 501.1396

Bis-2,3-(tert-butoxy)-5,6-bis(4-chlorophenyl)pyrazine-2,3-dicarboxylate may also be prepared by reacting 3-(tert-butoxycarbonyl)-5,6-bis(4-chlorophenyl)pyrazine-2-carboxylic acid with tert-butanol by methods known to those skilled in the art.

### Example 3

### 5,6-bis(4-chlorophenyl)-3-(4,4-dimethyl-4,5-dihydrooxazol-2-yl)-pyrazine-2-carboxylic acid tert-butylester

### Step A: 5,6-bis(4-chlorophenyl)-3-(2-hydroxy-1,1-dimethyl-ethylcarbamoyl)-pyrazine-2-carboxylic acid tert-butylester

5,6-bis(4-chlorophenyl)-3-(tert-butoxycarbonyl)-pyrazine-2-carboxylic acid (250 mg, 0.561 mmol) and HATU (320 mg, 0.842 mmol) were stirred in anhydrous pyridine (5 ml) for 2 h. 2-Methyl-2-amino-1-propanol (75 mg, 0.842 mmol) was added to this mixture. After 3 h no reaction could be detected. PyBOP (409 mg, 0.786 mmol) dissolved in anhydrous dichloromethane (1 ml) was added and the resulting mixture was stirred overnight at room temperature. The solvents were evaporated. The residue was dissolved in ethyl acetate and washed with 1N HCl, brine and sat. NaHCO₃ consecutively. The organic layer was dried (Na₂SO₄) and evaporated. Flash chromatography using a step gradient of hexanes/ethyl acetate 75:25, then 60:40 gave 5,6-bis(4-chlorophenyl)-3-(2-hydroxy-1,1-dimethyl-ethylcarbamoyl)-pyrazine-2-carboxylic acid tert-butylester (92 mg, 0.178 mmol, 32%) as a colorless foam.

¹H NMR (400 MHz, CDCl₃) δ 7.65 (s, 1H), 7.48-7.28 (m, 8H), 4.56 (t, 6.4 Hz, 1H), 3.73 (d, 6.4 Hz, 2H), 1.66 (s, 9H), 1.42 (s, 6H)

### Step B: 5,6-bis(4-chlorophenyl)-3-(4,4-dimethyl-4,5-dihydrooxazol-2-yl)-pyrazine-2-carboxylic acid tert-butylester

5,6-bis(4-chlorophenyl)-3-(2-hydroxy-1,1-dimethyl-ethylcarbamoyl)-pyrazine-2-carboxylic acid tert-butylester (91 mg, 0.176 mmol) was dissolved in dichloromethane (10 ml) and cooled to −78° C. DAST (31 μl, 0.234 mmol) was added dropwise and the solution was stirred at −78° C. for 90 min. K₂CO₃ (49 mg, 0.352 mmol) was added and the solution was allowed to reach room temperature. The reaction mixture was diluted with dichloromethane and extracted with sat. NaHCO₃. The aqueous phase was extracted twice with dichloromethane. The combined organic phases were dried (Na₂SO₄) and evaporated. Flash chromatography using a step gradient of hexanes/ethyl acetate 90:10, 85:15, then 80:20 gave 5,6-bis(4chlorophenyl)-3-(4,4-dimethyl-4,5-dihydrooxazol-2-yl)-pyrazine-2-carboxylic acid tert-butylester (48 mg, 0.096 mmol, 55%) as a colorless solid.

¹H NMR (400 MHz, CDCl₃) δ 7.46-7.28 (m, 8H), 4.19 (s, 2H), 1.65 (s, 9H), 1.42 (s, 6H) HRMS Calcd for [C₂₆H₂₅Cl₂N₃O₃+H]⁺: 499.1430. Found: 499.1389.

### Example 4

### 5,6-bis(4-chlorophenyl)-3-(3-oxa-1-azaspiro[4.4]non-1-en-2-yl)-pyrazine-2-carboxylic acid tert-butylester

### Step A: 5,6-bis(4-chlorophenyl)-3-(N-(1-hydroxymethyl-1-cyclopentyl)carbamoyl)-pyrazine-2-carboxylic acid tert-butylester

5,6-bis(4-chlorophenyl)-3-(tert-butoxycarbonyl)-pyrazine-2-carboxylic acid (250 mg, 0.561 mmol), cycloleucinol (97 mg, 0.842 mmol) and triethylamine (390 μl, 2.8 mmol) were suspended in dichloromethane (10 ml). Then PyBOP (438 mg, 0.842 mmol) in dichloromethane (5 ml) was added dropwise. The resulting mixture was stirred at room temperature overnight. The solution was poured into ethyl acetate and washed with 1N HCl, brine and sat. NaHCO₃. The organic phase was dried (Na₂SO₄) and evaporated. Flash chromatography using a step gradient hexanes/ethyl acetate 85:15, then 70:30 gave 5,6-bis(4-chlorophenyl)-3-(N-(1-hydroxymethyl-1-cyclopentyl)carbamoyl)-pyrazine-2-carboxylic acid tert-butylester (252 mg, 0.465 mmol, 83%) as a colorless foam.

¹H NMR (400 MHz, CDCl₃)δ 7.75 (s, 1H), 7.46-7.28 (m, 8H), 4.49 (t, 6.2 Hz, 1H), 3.78 (d, 6.4 Hz, 2H), 2.03-1.71 (m, 8H), 1.65 (s, 9H)

### Step B: 5,6-bis(4-chlorophenyl)-3-(3-oxa-1-azaspiro[4,4]non-1-en-2-yl)-pyrazine-2-carboxylic acid tert-butylester

5,6-bis(4-chlorophenyl)-3-(1-hydroxymethyl-cyclopentylcarbamoyl)-pyrazine-2-carboxylic acid tert-butylester (119 mg, 0.219 mmol) was dissolved in dichloromethane (10 ml) and cooled to −78° C. DAST (43 μl, 0.329 mmol) was added dropwise and the solution was stirred at −78° C. for 30 min. K₂CO₃ (91 mg, 0.658 mmol) was added and the solution was allowed to reach room temperature. The reaction mixture was diluted with ethyl acetate and washed with sat. NaHCO₃. The organic phase was dried (Na₂SO₄) and evaporated. Flash chromatography using hexanes/ethyl acetate 90:10 gave 5,6-bis(4-chlorophenyl)-3-(3-oxa-1-azaspiro[4.4]non-1-en-2-yl)-pyrazine-2-carboxylic acid tert-butylester (75 mg, 0.143 mmol, 65%) as a colorless solid.

¹H NMR (400 MHz, CDCl₃) δ 7.45-7.28 (m, 8H), 4.32 (s, 2H), 2.10-1.65 (m, 8H), 1.64 (s, 9 H)

HRMS Calcd for [C₂₈H₂₇Cl₂N₃O₃+H]⁺: 525.1587. Found: 525.1563.

### Example 5

### 5,6-bis(4-chlorophenyl)-3-(4-methyl-4,5-dihydrooxazol-2-yl):pyrazine-2-carboxylic acid tert-butylester

### Step A: 5,6-bis(4-chlorophenyl)-3-(N-(2-hydroxy-1-methylethyl)carbamoyl)-pyrazine-2-carboxylic acid tert-butylester

5,6-bis(4-chlorophenyl)-3-(tert-butoxycarbonyl)-pyrazine-2-carboxylic acid (400 mg, 0.898 mmol), DL-alaninol (101 mg, 1.347 mmol) and triethylamine (625 μl, 4.491 mmol) were dissolved in dichloromethane (10 ml). Then PyBOP (701 mg, 1.347 mmol) in dichloromethane (5 ml) was added dropwise. The resulting mixture was stirred at room temperature overnight. The solution was poured into ethyl acetate and washed with 1N HCl, brine and sat. NaHCO₃. The organic phase was dried (Na₂SO₄) and evaporated. Flash chromatography using a step gradient hexanes/ethyl acetate 85:15, 75:25 then 60:40 gave 5,6-bis(4-chlorophenyl)-3-(N-(2-hydroxy-1-methylethyl)carbamoyl)pyrazine-2-carboxylic acid tert-butylester (384 mg, 0.765 mmol, 85%) as a colorless solid.

¹H NMR (400 MHz, CDCl₃) δ 7.70 (d, 7.4 Hz, 1H), 7.52-7.29 (m, 8H), 4.26-4.28 (m, 1H), 3.82-3.76 (m, 1H), 3.71-3.65 (m, 1H), 1.67 (s, 9H), 1.31 (d, 6.8 Hz, 3H)

### Step B: 5,6-bis(4-chlorophenyl)-3-(4-methyl-4,5-dihydrooxazol-2-yl)-pyrazine-2-carboxylic acid tert-butylester

5,6-bis(4-chlorophenyl)-3-(2-hydroxy-1-methylethylcarbamoyl)-pyrazine-2-carboxylic acid tert-butylester (380 mg, 0.756 mmol) was dissolved in dichloromethane (10 ml) and cooled to −78° C. DAST (149 μl, 1.135 mmol) was added dropwise and the solution was stirred at −78° C. for 1 h. K₂CO₃ (314 mg, 2.269 mmol) was added and the solution was allowed to reach room temperature. The organic phase was washed with sat. NaHCO₃. The aqueous phase was extracted with DCM. The combined organic phases were dried (Na₂SO₄) and evaporated. Flash chromatography using a linear gradient of heptane/ethyl acetate 90:10 to 75:25 gave 5,6-bis(chlorophenyl)-3-(4-methyl-4,5-dihydrooxazol-2-yl)-pyrazine-2-carboxylic acid tert-butylester (230 mg, 0.474 mmol, 63%) as a colorless solid.

¹H NMR (400 MHz, CDCl₃) δ 7.47-7.28 (m, 8H), 4.62 (dd, 9.3 Hz, 8.0 Hz, 1H), 4.53-4.46 (m, 1H), 4.05 (dd, 8.1 Hz, 8.0 Hz, 1H), 1.64 (s, 9H), 1.41 (d, 6.4 Hz, 3H)

HRMS Calcd for [C₂₈H₂₃Cl₂N₃O₃+H]⁺: 485.1273. Found: 485.1284.

### Example 6

### 5,6-bis(4-chlorophenyl)-3-(4-methyloxazol-2-yl)-pyrazine-2-carboxylic acid tert-butylester

5,6-bis(4-chlorophenyl)-3-(4-methyl-4,5-dihydrooxazol-2-yl)-pyrazine-2-carboxylic acid tert-butylester (146 mg, 0.301 mmol) and DDQ (103 mg, 0.452 mmol) were dissolved in toluene (2 ml) in a microwave vessel with stirbar. The vessel was microwaved (temperature setting 150° C., holding time 10 min). The mixture was filtered through a plug of silica gel, eluted with toluene/EtOAc 9:1, then 8:2.

Product-containing fractions were purified by flash chromatography using heptane/ethyl acetate 9:1 as eluent. 5,6-bis(4-chlorophenyl)-3-(4-methyloxazol-2-yl)-pyrazine-2-carboxylic acid tert-butylester (16 mg, 0.0323 mmol, 10.7%) was isolated as colorless solid.

¹H NMR (400 MHz, CDCl₃) δ 7.58 (s, 1H), 7.49-7.29 (m, 8H), 2.28 (s, 3H), 1.62 (s, 9H)

HRMS Calcd for [C₂₅H₂₁Cl₂N₃O₃+H]⁺: 483.1117. Found: 483.1110.

### Example 7

### 5,6-bis(4-chlorophenyl)-3-(4-phenyl-4,5-dihydrooxazol-2:yl):pyrazine-2-carboxylic acid tert-butylester)

### Step A: 5,6-bis(4-chlorophenyl)-3-(2-hydroxy-1-phenylethylcarbamoyl)-pyrazine-2-carboxylic acid tert-butylester

5,6-bis(4-chlorophenyl)-3-(tert-butoxycarbonyl)-pyrazine-2-carboxylic acid (400 mg, 0.898 mmol), phenylglycinol (185 mg, 1.347 mmol) and triethylamine (630 μl, 4.5 mmol) were dissolved in DCM (10 ml). Then PyBOP (701 mg, 1.347 mmol) dissolved in 5 ml DCM, was added dropwise. The resulting mixture was stirred at room temperature overnight. The mixture was poured into ethyl acetate and washed with 1N HCl, brine and sat. NaHCO₃. The organic layer was dried (Na₂SO₄) and evaporated. Flash chromatography using a step gradient hexanes/ethyl acetate 80:20, then 60:40 gave 5,6-bis(4-chlorophenyl)-3-(2-hydroxy-1-phenylethylcarbamoyl)-pyrazine-2-carboxylic acid tert-butylester (412 mg, 0.730 mmol, 81%) as a colorless solid.

¹H NMR (400 MHz, CDCl₃) δ 8.28 (s, 1H), 7.49-7.26 (m, 13H), 5.31 (m, 1H), 3.86 (m, 2H), 1.65 (s, 9H)

### Step B: 5,6-bis(4-chlorophenyl)-3-(4:phenyl-4,5-dihydrooxazol-2-yl)-pyrazine-2-carboxylic acid tert-butylester)

5,6-bis(4-chlorophenyl)-3-(2-hydroxy-1-phenylethylcarbamoyl)-pyrazine-2-carboxylic acid tert-butylester (133 mg, 0.236 mmol) was dissolved in anhydrous DCM (10 ml) and cooled to −78° C. Then DAST (50 μl, 0.353 mmol) was added dropwise and the resulting mixture was stirred at −78° C. for 2 h. Then K₂CO₃ (98 mg, 0.707 mmol) was added and the reaction mixture was allowed to reach room temperature. Saturated NaHCO₃ was added, the phases separated and the aqueous phase extracted with DCM. The combined organic phases were dried (Na₂SO₄) and evaporated. Flash chromatography using a step gradient hexanes/ethyl acetate 90:10, then 85:15 gave 5,6-bis(4-chlorophenyl)-3-(4-phenyl-4,5-dihydrooxazol-2-yl)-pyrazine-2-carboxylic acid tert-butylester (110 mg, 0.201 mmol, 85%) as a colorless solid.

¹H NMR (400 MHz, CDCl₃) δ 8.28 (s, 1H), 7.51-7.27 (m, 13H), 5.51 (dd, 8.9 Hz, 8.6 Hz, 1 H), 4.90 (dd, 8.5 Hz, 8.4 Hz, 1H), 4.38 (t, 8.4 Hz, 1H), 1.54 (s, 9H)

HRMS Calcd for [C₃₀H₂₅Cl₂N₃O₃+H]⁺: 547.1430. Found: 547.1411.

### Example 8

### 5,6-bis(4-chlorophenyl)-3-(4-phenyloxazol-2-yl)-pyrazine-2-carboxylic acid tert-butylester

5,6-bis(4-chlorophenyl)-3-(4-phenyl-4,5-dihydrooxazol-2-yl)-pyrazine-2-carboxylic acid tert-butylester (54 mg, 0.099 mmol) and DDQ (34 mg, 0.148 mmol) were dissolved in toluene (2 ml) in a microwave vessel with stirbar. The vessel was microwaved for 10 min, temperature setting 150° C., no holding time. It took 5 minutes for the system to come to 150° C., so the effective heating time was 5 min. 200 μl of ethyl acetate were added to the reaction mixture, which was filtered through a plug of silica and washed with toluene/ethyl acetate 9:1. Product containing fractions were further purified by flash chromatography using heptanes/ethyl acetate 9:1 as eluent. 5,6-bis(4-chlorophenyl)-3-(4-phenyloxazol-2-yl)-pyrazine-2-carboxylic acid tert-butylester (15 mg, 0.027 mmol, 28%) was isolated as colorless solid.

¹H NMR (400 MHz, CDCl₃) δ 8.13 (s, 1H), 7.82-7.85 (m, 2H), 7.52-7.31 (m, 11H), 1.59 (s, 9H)

HRMS Calcd for [C₃₀H₂₃Cl₂N₃O₃+H]⁺: 545.1274. Found: 545.1271.

### Example 9

### 5,6-bis(4-chlorophenyl)-3-(5-phenyl-4,5-dihydrooxazol-2-yl)-pyrazine-2-carboxylic acid tert-butylester

### Step A: 5,6-bis(4-chlorophenyl)-3-(N-(2-hydroxy-2-phenylethyl)carbamoyl)-pyrazine-2-carboxylic acid tert-butylester

5,6-bis(4-chlorophenyl)-3-(tert-butoxycarbonyl)-pyrazine-2-carboxylic acid (400 mg, 0.898 mmol), 2-amino-1-phenylethanol (185 mg, 1.347 mmol) and triethylamine (630 μl, 4.5 mmol) were dissolved in DCM (10 ml). Then PyBOP (701 mg, 1.347 mmol) dissolved in 5 ml DCM, was added dropwise. The resulting mixture was stirred at room temperature overnight. The mixture was poured into ethyl acetate and extracted with 1 N HCl, brine and sat. NaHCO₃. The organic layer was dried (Na₂SO₄) and evaporated. Flash chromatography using a step gradient hexanes/ethyl acetate 80:20, 75:25 then 70:30 gave 5,6-bis(4-chlorophenyl)-3-(N-(2-hydroxy-2-phenylethyl)carbamoyl)pyrazine-2-carboxylic acid tert-butylester (457 mg, 0.810 mmol, 90%) as a colorless solid.

¹H NMR (400 MHz, CDCl₃) δ 8.01-8.00 (m, 1H), 7.47-7.28 (m, 13H), 4.93-4.95 (m, 1H), 3.91-3.84 (m, 1H), 3.60-3.53 (m, 1H), 3.31 (s, H), 1.66 (s, 9H)

### Step B: 5,6-bis(4-chlorophenyl)-3-(5-phenyl-4,5-dihydrooxazol-2-yl)-pyrazine-2-carboxylic acid tert-butylester

5,6-bis(4-chlorophenyl)-3-(2-hydroxy-2-phenylethylcarbamoyl)-pyrazine-2-carboxylic acid tert-butylester (288 mg, 0.51 mmol) and Burgess' Reagent (134 mg, 0.561 mmol) were dissolved in THF (10 ml) and the resulting mixture was heated to 70° C. for 30 min. The solvent was evaporated. The residue was purified by flash chromatography using a step gradient of heptanes/ethyl acetate 85:15, then 80:20 to give 5,6-bis(4-chlorophenyl)-3-(5-phenyl-4,5-dihydrooxazol-2-yl)-pyrazine-2-carboxylic acid tert-butylester (170 mg, 0.311 mmol, 61%) as a colorless solid.

¹H NMR (400 MHz, CDCl₃) δ 7.51-7.26 (m, 13H), 5.77 (dd, 8.4 Hz, 8.3 Hz, 1H), 4.58 (dd, 15.3 Hz, 10.2 Hz, 1H), 4.09 (dd, 15.5 Hz, 8.2 Hz, 1H), 1.56 (s, 9H)

HRMS Calcd for [C₃₀H₂₅Cl₂N₃O₃+H]⁺: 547.1430. Found: 547.1427.

### Example 10

### tert-butyl 5,6-bis(4-chlorophenyl)-3-(2H-tetrazol-2-ylmethyl)pyrazine-2-carboxylate

### Step A Ethyl 5,6-bis(4-chlorophenyl)-3-(2H-tetrazol-2-ylmethyl)pyrazine-2-carboxylate and ethyl 5,6-bis(4-chlorophenyl)-3-(1H-tetrazol-1-ylmethyl)pyrazine-2-carboxylate

To a solution of ethyl 5,6-bis(4-chlorophenyl)-3-(hydroxymethyl)pyrazine-2-carboxylate (0.32 g, 0.80 mmol) in tetrahydrofuran (10 ml) were added 1H-tetrazole (84 mg, 1.20 mmol) and triphenylphosphine (0.25 g, 0.96 mmol). Upon cooling to 0° C., diethyl azodicarboxylate (0.16 ml, 0.84 mmol) was added dropwise. The reaction mixture was stirred at 0° C. for 1 h. The solvent was removed under reduced pressure and separation by prepHPLC gave two isomers:ethyl 5,6-bis(4-chlorophenyl)-3-(2H-tetrazol-2-ylmethyl)pyrazine-2-carboxylate (180 mg, 50%) as a white solid.

¹H NMR (400 MHz, CDCl3) δ 8.60 (s, 1H), 7.46 (d, 2H), 7.33 (d, 2H), 7.22 (d, 4H), 6.52 (s, 2H), 4.53 (q, 2H), 1.47 (t, 3H).

MS m/z 455 (M+H)⁺.

and ethyl 5,6-bis(4-chlorophenyl)-3-(1H-tetrazol-1-ylmethyl)pyrazine-2-carboxylate (88 mg, 24%) as a white solid.

¹H NMR (400 MHz, CDCl₃) δ 8.89 (s, 1H), 7.46 (d, 2H), 7.34 (d, 2H), 7.27 (d, 4H), 6.29 (s, 2H), 4.55 (q, 2H), 1.49 (t, 3H).

MS m/z 455 (M+H)⁺.

### Step B 5,6-bis(4-chlorophenyl)-3-(2H-tetrazol-2-ylmethyl)pyrazine-2-carboxylic acid

To a solution of ethyl 5,6-bis(4-chlorophenyl)-3-(2H-tetrazol-2-ylmethyl)pyrazine-2-carboxylate (200 mg, 0.44 mmol) in acetonitrile were added a solution of lithium hydroxide (42 mg, 1.76 mmol) in water (3.0 ml) and tetrahydrofuran (3 ml). The reaction solution was stirred in room temperature overnight. The solvent was removed under reduced pressure and water was added to the residue. The aqueous phase was acidified by adding 1 M HCl and extracted with dichloromethane and the collected organic phases were evaporated to give the title compound (187 mg, 100%) as a white solid.

MS m/z 427 (M+H)⁺.

### Step C tert-butyl 5,6-bis(4-chlorophenyl)-3-(2H-tetrazol-2-ylmethyl)pyrazine-2-carboxylate

5,6-bis(4-chlorophenyl)-3-(2H-tetrazol-2-ylmethyl)pyrazine-2-carboxylic acid (104 mg, 0.24 mmol) was suspended in toluene and heated to 77° C. N,N-dimethylformamide di-tert-butyl acetal (198 mg, 0.97 mmol) was carefully added, and the reaction solution was heated at reflux overnight. The reaction mixture was cooled, and water and diethyl ether was added. The organic phase was separated and washed with NaHCO₃ and water before drying with Na₂SO₄. The solvent was removed under reduced pressure and preparatory HPLC gave the title compound (55 mg, 47%) as a solid.

¹H NMR (400 MHz, CDCl3) δ 8.59 (s, 1H), 7.46 (d, 2H), 7.31 (d, 2H), 7.21 (s, 4H), 6.46 (s, 2H), 1.65 (s, 9H).

MS m/z 483 (M+H)⁺.

Preparation of Intermediates

### a) 1,2-bis(4-chlorophenyl)-2-hydroxyethanone

To 4-chlorobenzaldehyde (140.6 g, 1 mol) in ethanol (130 ml) was added a solution of sodium cyanide (10.6 g, 0.216 mol) in water (105 ml). The mixture was heated at reflux for 2.5 h and then extracted with DCM. The organic phase was washed with sodium bisulfite solution and the solvent was evaporated in vacuo. The compound was isolated by crystallization from diethyl ether/heptane. 48 g, 34%.

¹H NMR (400 MHz) δ 7.82 (d, 2H), 7.38 (d, 2H), 7.30 (d, 2H), 7.24 (d, 2H), 5.87 (s, 1H), 4.47 (s, 1H).

MS m/z 279, 281 (Me

### b) 1,2-bis(4-chlorophenyl)ethane-1,2-dione

1,2-bis(4-chlorophenyl)-2-hydroxyethanone, (90 g, 0.320 mol) and nitric acid (170 ml) were heated at 100° C. until the evolution of nitrogen oxides ceased after 4 hours. The reaction mixture was cooled, and water (250 ml) was carefully added. The crude product was filtered, washed several times with water and dried under reduced pressure to give a yellow solid (40.4 g, 45%).

¹H NMR (500 MHz) δ 7.94 (d, 4H), 7.53 (d, 4H).

### c) 5,6-bis(4-chlorophenyl)pyrazine-2,3-dicarbonitrile

1,2-bis(4-chlorophenyl)ethane-1,2-dione, (20 g, 71.65 mmol), diaminomaleonitrile (8.5 g, 78.82 mmol) and acetic acid (6 ml) in ethanol (140 ml) and water (93 ml) were heated at 75° C. overnight. The reaction mixture was cooled, and water was added. The precipitate was filtered and washed with ethanol and then ether. The crude product was dissolved in DCM and treated with activated charcoal, then filtered through celite. After evaporation, a solid was formed and recrystallized from DCM/ethanol to give a pale yellow solid (17.3 g, 69%).

¹H NMR (400 MHz) δ 7.49 (d, 4H), 7.38 (d, 4H).

### d) 5,6-bis(4-chlorophenyl)pyrazine-2,3-dicarboxylic acid

To 5,6-bis(4-chlorophenyl)pyrazine-2,3-dicarbonitrile, (16.3 g, 46.28 mmol) and KOH (26 g, 463 mmol) in water (84 ml) was added hydrogen peroxide (35%, 19 ml) followed by a few drops of nonanol to reduce foaming. The reaction mixture was heated at reflux for 2 h, cooled and washed once with diehtyl ether and acidified to pH 4 with 2M HCl. The precipitate was collected through a filter, washed with water and dried under reduced pressure to give the crude product. The crude product was converted to dimethyl ester by refluxing with hydrogen chloride/methanol (100 ml) and purified by HPLC, giving 12.85 g of the methyl ester. The resulting methyl ester was treated with lithium hydroxide (2.95 g, 0.123 mmol) in acetonitrile (140 ml) and water (90 ml) at ambient temperature for 1.5 h. The acetonitrile was removed under reduced pressure and the aqueous solution was washed once with diethyl ether. Acidification with hydrochloric acid (2M) and filtration gave the title compound (11.8 g, 66% mmol) as a pale yellow solid.

¹H NMR (400 MHz) δ 7.51 (d, 4H), 7.41 (d, 4H). MS m/z 389, 391 (M+H)⁺.

### e) 2,3-bis(4-chlorophenyl)furo[3,4-b]pyrazine-5,7-dione

5,6-bis(4-chlorophenyl)pyrazine-2,3-dicarboxylic acid (6.7 g, 17.30 mmol) and acetyl chloride (20 ml) were heated at reflux overnight. The acetyl chloride was removed under reduced pressure to give the title compound (6.2 g, 97%) as a pale yellow solid.

¹H NMR (400 MHz) δ 7.51 (d, 4H), 7.41 (d, 4H).

### f) 5,6-bis(4-chlorophenyl)-3-(tert-butoxycarbonyl)-pyrazine-2-carboxylic acid

To a solution of 2,3-bis(4-chlorophenyl)furo[3,4-b]pyrazine-5,7-dione, (877 mg, 2.36 mmol) in acetonitrile (15 ml) were added tert-butanol (876 mg, 11.8 mmol) and DMAP (346 mg, 2.8 mmol). After 30 minutes the solvent was removed in vacuo and the residue was dissolved in DCM. Washed with 2 M potassium hydrogen sulfate and water followed by drying (magnesium sulfate), filtration and evaporation of the solvent gave a residue which was purified by HPLC to give the title compound (431 mg, 41%).

¹H NMR (400 MHz) δ 7.35-7.17 (m, 8H), 1.57 (s, 9H)

MS m/z 445 (M+H)⁺, 443 (M-H)—.

Pharmacological Activity

Compounds of the present invention are active against the receptor product of the CB1 gene. The compounds of the present invention are active at the CB1 receptor (IC50<1 micromolar). Most preferred compounds have IC50<200 nanomolar. The affinity of the compounds of the invention for central cannabinoid receptors is demonstrable in methods described in Devane et al., Molecular Pharmacology, 1988, 34,605 or those described in WO01/70700 or EP 656354. Alternatively the assay may be performed as follows.

10 μg of membranes prepared from cells stably transfected with the CB1 gene were suspended in 2001 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x)UD)) and the IC50 value determined as the concentration required to give half maximal inhibition of GTPγS binding under the conditions used.

For instance, example 5, (5,6-bis(4-chlorophenyl)-3-(4-methyl-4,5-dihydrooxazol-2-yl)-pyrazine-2-carboxylic acid tert-butylester) exhibits an IC50 (hCB1) at 1.8 nM.

The compounds of the present invention may provide additional benefits in terms of potency, selectivity, bioavailability, half-life in plasma, blood brain permeability, plasma protein binding or solubility compared to representative reference CB1 antagonists/inverse agonist agents.

1. A compound of formula (I): 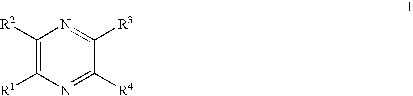
or a pharmaceutically acceptable salt thereof, in which
R¹ and R² independently represent phenyl, thienyl or pyridyl each of which is independently optionally substituted by one or more groups represented by Z;
Z represents a C₁₋₈alkyl group, a C₁₋₆alkoxy group, hydroxy, halo, trifluoromethyl, trifluoromethylthio, trifluoromethoxy, trifluoromethylsulphonyl, nitro, mono or di C₁₋₃alkylamido, C₁₋₃alkylthio, C₁₋₃alkylsulphonyl, C₁₋₃alkylsulphonyloxy, C₁₋₃alkoxycarbonyl, carboxy, cyano, carbamoyl, mono or di C₁₋₃alkyl carbamoyl, sulphamoyl, acetyl, an aromatic heterocyclic group which is optionally substituted by one or more halo, C₁₋₄alkyl, trifluoromethyl or trifluoromethoxy, or Z represents a saturated or partially unsaturated 5- to 8membered heterocyclic group containing one or more heteroatoms selected from nitrogen, oxygen or sulphur wherein the heterocyclic group is optionally substituted by one or more C₁₋₃alkyl, hydroxy, fluoro, benzyl or an amino group —NR^{x}R^{y} in which R^{x} and R^{y} independently represent H or C₁₋₄alkyl;
R³ and R⁴ independently represent a group of formula (CH₂)ₙCOOR⁷ in which n is 0, 1, 2, 3 or 4;
and R⁷ represents a C₄₋₁₂alkyl group, a C₃₋₁₂cycloalkyl group or a (C₃₋₁₂cycloalkyl)C₁₋₃alkyl- group each of which is optionally substituted by one or more of the following: a C₁₋₆alkyl, fluoro, amino or hydroxyl group, or R⁷ represents a group —(CH₂)ₐphenyl in which a is 0, 1, 2, 3 or 4 and the phenyl group is optionally substituted by one or more groups represented by Z which may be the same or different or R⁷ represents a saturated or partially unsaturated 5- to 8 membered heterocyclic group containing one or more of the following: oxygen, sulphur or nitrogen; wherein the heterocyclic group is optionally substituted by one or more C₁₋₃alkyl, C₁₋₃acyl, hydroxy, amino or benzyl groups; or
R³ and R⁴ independently represent a group of formula —(CH₂)ₒ—O—(CH₂)ₚ—R⁵ in which o and p independently represent an integer 0, 1, 2, 3 or 4, with the proviso that neither R³ or R⁴ is methoxy, and R⁸ represents a C₁₋₁₂alkyl group or R⁸ represents phenyl optionally independently substituted by one or more Z groups or R⁸ represents an aromatic heterocyclic group or a saturated or partially unsaturated 5- to 8membered heterocyclic group containing one or more of the following: oxygen, sulphur or nitrogen wherein each of these rings is optionally substituted by one or more groups represented by Z which may be the same or different; or
R³ and R⁴ independently represent a C₁₋₁₂alkyl group optionally substituted by one or more fluoro, hydroxy, or amino groups, provided that if R³ is C₁₋₄alkyl then R⁴ cannot be C₁₋₄alkyl or q cannot be 0 in R⁴, or
R³ and R⁴ independently represent a group of formula —(CH₂)_{q}R⁹ in which q is 0, 1, 2, 3 or 4, provided that if q is 0 in R³ then q cannot be 0 in R⁴, and if q is 0 in R⁴ then q cannot be 0 in R³, R⁹ represents a C₃₋₁₂cycloalkyl group, phenyl, an aromatic heterocyclic group or a saturated or partially unsaturated 5- to 12membered heterocyclic group containing one or more of the following: oxygen, sulphur or nitrogen, wherein each of these rings is optionally substituted by one or more groups represented by Z which may be the same or different or each of these rings is substituted by phenyl which is optionally substituted by one or more C₁₋₄alkyl, C₁₋₄alkoxy, hydroxy, halo or trifluoromethyl; or
R³ and R⁴ independently represent a group of formula —(CH₂), —O—(CO)—R¹⁰ in which m represents an integer 0, 1, 2, 3 or 4, in which R¹⁰ represents a C₁₋₁₂alkyl group optionally substituted by one or more fluoro, hydroxy, or amino groups or R¹⁰ represents a group of formula —(CH₂)_{q}R⁹; or
R³ and R⁴ are identical and represent a group of formula CONR¹¹R¹² in which R¹¹ and R¹² independently represent a C₁₋₆alkyl group; an (amino)C₁₋₄alkyl- group in which the amino is optionally substituted by one or more C₁₋₃alkyl groups; a (C₃₋₁₂cycloalkyl)(CH₂)_{g}— group wherein g is 0, 1, 2 or 3, wherein the cycloalkyl is optionally substituted by one or more fluoro, hydroxy, C₁₋₃alkyl, C₁₋₃alkoxy, C₁₋₃alkoxycarbonyl, trifluoromethyl, amino or trifluoromethoxy groups; a group —(CH₂)ᵣ(phenyl)s in which r is 0, 1, 2, 3 or 4, s is 1 when r is 0 otherwise s is 1 or 2 and the phenyl groups are optionally independently substituted one or more groups represented by Z; naphthyl; anthracenyl; a saturated or partially unsaturated 5- to 8 -membered heterocyclic group containing one or more heteroatoms selected from nitrogen, oxygen or sulphur wherein the heterocyclic group is optionally substituted by one or more C₁₋₃alkyl, hydroxy, fluoro, trifluoromethyl, benzyl or an amino group —NR^{x}R^{y} in which R^{x} and R^{y} independently represent H or C₁₋₄alkyl; 1-adamantylmethyl; a group —(CH₂)ₜHet in which t is 0, 1, 2, 3 or 4, and the alkylene chain is optionally substituted by one or more C₁₋₃alkyl groups and Het represents an aromatic heterocyclic group optionally substituted by one, two or three groups selected from a C₁₋₅alkyl group, a C₁₋₅alkoxy group or halo; or R¹¹ represents H and R¹² is as defined above; or R¹¹ and R¹² together with the nitrogen atom to which they are attached represent a saturated or partially unsaturated 5- to 8membered heterocyclic group containing one nitrogen and optionally one of the following: oxygen, sulphur or an additional nitrogen; wherein the heterocyclic group is optionally substituted by one or more C₁₋₃alkyl, hydroxy, fluoro, trifluoromethyl, trifluoromethoxy, benzyl, C₁₋₆alkanoyl or an amino group —NR^{x}R^{y} in which R^{x} and R^{y} independently represent H or C₁₋₄alkyl;
with the provisos that
1) when R³ and R⁴ are both a group of formula CONR¹¹R¹² then they do not represent carbamoyl, or mono or di C₁₋₃alkylcarbamoyl, and
2) when R¹, R² and R³ each represent phenyl then R⁴ is not benzyl, and
3) when one of R³ or R⁴ is C₁₋₄alkyl then the other is not a group —(CH₂)_{q}R⁹ in which q is 0.

2. A compound according to claim 1, wherein R¹ and R² are phenyl optionally substituted by one or more groups Z.

3. A compound according to claim 1, wherein R¹ and R² are both 4-chlorophenyl.

4. A compound according to claim 1, wherein R³ and R⁴ independently represent a group of formula COOR⁷ in which R⁷ is a C₄₋₈alkyl group.

5. A compound according to claim 1, wherein R³ represents a group of formula COOR⁷ in which R⁷ is a C₄₋₈alkyl group and R⁴ represents a group of formula —(CH₂)ₒ—O—(CH₂)ₚ—R⁸ in which o and p independently represent an integer 0, 1, 2, 3 or 4, and R⁸ represents phenyl optionally independently substituted by one or more Z groups.

6. A compound according to claim 1, wherein R³ and R⁴ both represent a group of formula CONR¹¹R¹² in which R¹¹ and R¹² together with the nitrogen atom to which they are attached represent piperidino.

7. A compound according to claim 1, wherein R³ represents a group of formula COOR⁷ in which R⁷ is a C₄₋₈alkyl group and R⁴ represents a group of formula R³ and R⁴ independently represent a group of formula —(CH₂)ₘ—O—(CO)—R¹⁰ in which m represents an integer 0, 1, 2, 3 or 4, in which R¹⁰ represents a C₁₋₁₂alkyl group optionally substituted by one or more fluoro, hydroxy, or amino groups or R¹⁰ represents phenyl optionally substituted by one or more groups represented by Z which may be the same or different.

8. A compound according to claim 1, wherein R³ and R⁴ are identical.

9. A compound according to claim 1 as represented by formula II: 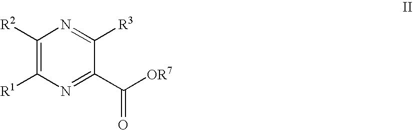
in which R¹ and R² are both 4-chlorophenyl;
R³ represents dihydrooxazolyl, (3-oxa-1-azaspiro[4.4]nonenyl), oxazolyl or tetrazol-2-ylmethyl optionally substituted by phenyl or a C₁₋₄alkyl group; and
R⁷ represents a C₄₋₁₂alkyl group, a C₃₋₁₂cycloalkyl group or a (C₃₋₁₂cycloalkyl)C₁₋₃alkyl- group each of which is optionally substituted by one or more of the following: a C₁₋₆alkyl fluoro, amino or hydroxyl grout.

10. A compound selected from:
2,3-bis(4-chlorophenyl)-5,6-bis(piperidin-1-ylcarbonyl)pyrazine,
bis-2,3-(tert-butoxy)-5,6-bis(4-chlorophenyl)pyrazine-2,3-dicarboxylate,
5,6-bis(4-chlorophenyl)-3-(4,4-dimethyl-4,5-dihydrooxazol-2-yl)-pyrazine-2-carboxylic acid tert-butylester,
5,6-bis(4-chlorophenyl)-3-(3-oxa-1-azaspiro[4.4]non-1-en-2-yl)-pyrazine-2-carboxylic acid tert-butylester,
5,6-bis(4-chlorophenyl)-3-(4-methyl-4,5-dihydrooxazol-2-yl)-pyrazine-2-carboxylic acid tert-butylester,
5,6-bis(4-chlorophenyl)-3-(4-methyloxazol-2-yl)-pyrazine-2-carboxylic acid tert-butylester,
5,6-bis(4-chlorophenyl)-3-(4-phenyloxazol-2-yl)-pyrazine-2-carboxylic acid tert-butylester,
5,6-bis(4-chlorophenyl)-3-(5-phenyl-4,5-dihydrooxazol-2-yl)-pyrazine-2-carboxylic acid tert-butylester, or
tert-butyl 5,6-bis(4-chlorophenyl)-3-(2H-tetrazol-2-ylmethyl)pyrazine-2-carboxylate, or a pharmaceutically acceptable salt thereof.

11. (canceled)

12. A pharmaceutical formulation comprising a compound of claim 1 and a pharmaceutically acceptable adjuvant, diluent or carrier.

13-14. (canceled)

15. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, neurological disorders, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound according to any one of claims 1 or 9-10 or a formulation of claim 12 to a patient in need thereof.

16. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, neurological disorders, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound of formula Ia, or a pharmaceutically acceptable salt thereof, to a patient in need thereof, wherein Formula Ia has the formula: 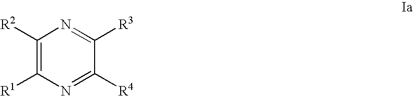
in which R¹ and R² independently represent phenyl, thienyl or pyridyl each of which is independently optionally substituted by one or more groups represented by Z;
Z represents a C₁₋₈alkyl group, a C₁₋₆alkoxy group, hydroxy, halo, trifluoromethyl, trifluoromethylthio, trifluoromethoxy, trifluoromethylsulphonyl, nitro, mono or di C₁₋₃alkylamido, C₁₋₃alkylsulphonyl, C₁₋₃alkylsulphonyloxy, C₁₋₃alkoxycarbonyl, carboxy, cyano, carbamoyl, mono or di C₁₋₃alkyl carbamoyl, sulphamoyl, acetyl, an aromatic heterocyclic group which is optionally substituted by one or more halo, C₁₋₄alkyl, trifluoromethyl or trifluoromethoxy, or Z represents a saturated or partially unsaturated 5- to 8membered heterocyclic group containing one or more heteroatoms selected from nitrogen, oxygen or sulphur wherein the heterocyclic group is optionally substituted by one or more C₁₋₃alkyl, hydroxy, fluoro, benzyl or an amino group —NR^{x}R^{y} in which R^{x} and R^{y} independently represent H or C₁₋₄alkyl;
R³ and R⁴ independently represent a group of formula (CH₂)ₙCOOR⁷ in which n is 0, 1, 2, 3 or 4; and R⁷ represents a C₁₋₁₂alkyl group, a C₃₋₁₂cycloalkyl group or a (C₃₋₁₂cycloalkyl)C₁₋₃alkyl- group each of which is optionally substituted by one or more of the following: a C₁₋₆alkyl, fluoro, amino or hydroxyl group, or R⁷ represents a group —(CH₂)ₐphenyl in which a is 0, 1, 2, 3 or 4, and the phenyl group is optionally substituted by one or more groups represented by Z which may be the same or different or R⁷ represents a saturated or partially unsaturated 5- to 8membered heterocyclic group containing one or more of the following: oxygen, sulphur or nitrogen; wherein the heterocyclic group is optionally substituted by one or more C₁₋₃alkyl, C₁₋₃acyl, hydroxy, amino or benzyl groups; or
R³ and R⁴ independently represent a group of formula —(CH₂)ₒ—O—(CH₂)ₚ—R⁸ in which o and p independently represent an integer 0, 1, 2, 3 or 4, and R⁸ represents a C₁₋₁₂alkyl group or R⁸ represents phenyl optionally independently substituted by one or more Z groups or R⁸ represents an aromatic heterocyclic group or a saturated or partially unsaturated 5- to 8-membered heterocyclic group containing one or more of the following: oxygen, sulphur or nitrogen wherein each of these rings is optionally substituted by one or more groups represented by Z which may be the same or different; or
R³ and R⁴ independently represent a C₁₋₁₂alkyl group optionally substituted by one or more fluoro, hydroxy, or amino groups; or
R³ and R⁴ independently represent a group of formula —(CH₂)_{q}R⁹ in which q is 0, 1, 2, 3 or 4, and R⁹ represents a C₃₋₁₂cycloalkyl group, phenyl, an aromatic heterocyclic group or a saturated or partially unsaturated 5- to 8membered heterocyclic group containing one or more of one following: oxygen, sulphur or nitrogen wherein each of these rings is optionally substituted by one or more groups represented by Z which may be the same or different; or
R³ and R⁴ independently represent a group of formula —(CH₂)ₘ—O—(CO)—R¹⁰ in which m represents an integer 0, 1, 2, 3 or 4, in which R¹⁰ represents a C₁₋₁₂alkyl group optionally substituted by one or more fluoro, hydroxy, or amino groups or R¹⁰ represents a group of formula —(CH₂)_{q}R⁹; or
R³ and R⁴ independently represent a group of formula CONR¹¹R¹² in which R¹¹ and R¹² independently represent a C₁₋₆alkyl group; an (amino)C₁₋₄alkyl- group in which the amino is optionally substituted by one or more C₁₋₃alkyl; a (C₃₋₁₂cycloalkyl)(CH₂)_{g}— group wherein g is 0,1, 2 or 3, wherein the cycloalkyl is optionally substituted by one or more fluoro, hydroxy, C₁₋₃alkyl, C₁₋₃alkoxy, C₁₋₃alkoxycarbonyl, trifluoromethyl, amino or trifluoromethoxy groups; a group —(CH₂)ᵣ(phenyl)ₛ in which r is 0, 1, 2, 3 or 4, s is 1 when r is 0, otherwise s is 1 or 2 and the phenyl groups are optionally independently substituted one or more groups represented by Z; naphthyl; anthracenyl; a saturated or partially unsaturated 5- to 8membered heterocyclic group containing one or more heteroatoms selected from nitrogen, oxygen or sulphur wherein the heterocyclic group is optionally substituted by one or more C₁₋₃alkyl, hydroxy, fluoro, trifluoromethyl, benzyl or an amino group —NR^{x}R^{y} in which R^{x} and R^{y} independently represent H or C₁₋₄alkyl; 1-adamantylmethyl; a group —(CH₂)ₜHet in which t is 0, 1, 2, 3 or 4, and the alkylene chain is optionally substituted by one or more C₁₋₃alkyl groups and Het represents an aromatic heterocyclic group optionally substituted by one, two or three groups selected from a C₁₋₅alkyl group, a C₁₋₅alkoxy group or halo; or R¹¹ represents H and R¹² is as defined above; or R¹¹ and R¹² together with the nitrogen atom to which they are attached represent a saturated or partially unsaturated 5- to 8membered heterocyclic group containing one nitrogen and optionally one of the following: oxygen, sulphur or an additional nitrogen; wherein the heterocyclic group is optionally substituted by one or more C₁₋₃alkyl, hydroxy, fluoro, trifluoromethyl, trifluoromethoxy, benzyl, C₁₋₆alkanoyl or an amino group —NR^{x}R^{y} in which R^{x} and R^{y} independently represent H or C₁₋₄alkyl;
with the proviso that when one of R³ and R⁴ is a C₁₋₃alkyl group, a C₁₋₃alkoxymethyl group, trifluoromethyl, a hydroxyC₁₋₃alkyl group, C₁₋₃alkoxycarbonyl, carboxy, carbamoyl, or mono or di C₁₋₃alkylcarbamoyl then the other does not represent a group of formula CONR¹¹R¹².

17. A method for the treatment of obesity comprising administering a pharmacologically effective amount of a compound of any one of claims 1 or 9-10 or a formulation of claim 12 to a patient in need thereof.
